(19) United States
(12) Patent Application Publication
(10) Pub. No.: US 2015/0283145 A1
(43) Pub. Date: Oct. 8, 2015

(54) TREATMENT OF HYPERPROLIFERATIVE AND PRE-CANCEROUS SKIN DISEASES USING AN INHIBITOR OF CBP/CATENIN

(71) Applicant: PRISM Pharma Co., Ltd., Kanagawa (JP)

(72) Inventors: Hiroyuki Kouji, Kanagawa (JP); Takenao Odagami, Kanagawa (JP)

(73) Assignee: PRISM Pharma Co., Ltd., Kanagawa (JP)

(21) Appl. No.: 14/436,672

(22) PCT Filed: Oct. 21, 2013
(86) PCT No.: PCT/JP2013/079055

§ 371 Date: Apr. 17, 2015

Related U.S. Application Data
(60) Provisional application No. 61/716,098, filed on Oct. 19, 2012.

(51) Int. Cl. A61K 31/53

(52) U.S. Cl. CPC A61K 31/53

(57) ABSTRACT

The present disclosure relates generally to alpha-helix mimetic structures and specifically to alpha-helix mimetic structures that are inhibitors of β-catenin. The disclosure also relates to applications in the treatment of hyperproliferative and pre-cancerous skin conditions including actinic keratosis and psoriasis, and pharmaceutical compositions comprising such alpha helix mimetic β-catenin inhibitors.

### CROSS-REFERENCE TO RELATED APPLICATIONS

This application claims priority to U.S. provisional application 61/716,098, filed Oct. 19, 2012, which is incorporated herein in its entirety.

### BACKGROUND OF THE DISCLOSURE

Wnt/β-catenin signaling is emerging as a forerunner for its critical roles in many facets of human biology. This signaling pathway has roles in embryogenesis, organogenesis, and maintaining tissue and organ homeostasis, and also in pathological conditions such as cancer and other human disorders such as inflammatory disorders and fibrosis. It is also integral in several physiological events such as differentiation, proliferation, survival, oxidative stress, morphogenesis, and others. However, aberrant activation of this pathway is also evident in multiple pathological conditions.

Psoriasis is a skin condition characterized by hyperproliferation of skin cells, pruritis/itching, and areas of inflamed tissue. Psoriasis is characterized by marked changes in keratinocyte growth and differentiation, and there is evidence for altered Wnt signaling in the disease. (J. Invest. Dermatol. 130(7):1849-59, 2010).

Actinic keratosis (also called “solar keratosis” and “senile keratosis”) is a premalignant condition of thick, scaly, or crusty patches of skin.

Wei et al. (Arthritis Rheum. 63(6):1707-17, 2011) cultured human keratinocytes with high proliferative potential, and found that such putative epidermal stem cells expressed a higher level of noncadherin-associated beta-catenin than populations enriched for keratinocytes of lower proliferative potential. To investigate the physiological significance of this, Wei et al. introduced a series of beta-catenin constructs into keratinocytes via retroviral infection. Full-length beta-catenin and a mutant containing only nine armadillo repeats had little effect on proliferative potential in culture, the full-length protein being rapidly degraded. However, expression of stabilized, N-terminally truncated beta-catenin increased the proportion of putative stem cells to almost 90% of the proliferative population in vitro without inducing malignant transformation, and relieved the differentiation stimulatory effect of overexpressing the E-cadherin cytoplasmic domain. Conversely, beta-catenin lacking armadillo repeats acted as a dominant negative mutant and stimulated exit from the stem cell compartment in culture.

Wei et al. found that the positive and negative effects of the beta-catenin mutants on proliferative potential were independent of effects on cell-cycle kinetics, overt terminal differentiation or intercellular adhesion, and correlated with stimulation or inhibition of transactivation of a TCF/LEF reporter in basal keratinocytes. Wei et al. concluded that the elevated level of cytoplasmic beta-catenin in those keratinocytes with characteristics of epidermal stem cells contributed to their high proliferative potential. The studies of Wei et al. provide evidence for a role of beta-catenin signaling in hyperproliferative and pre-cancerous skin conditions including actinic keratosis and psoriasis.

### BRIEF SUMMARY OF THE DISCLOSURE

This disclosure presents methods of treating actinic keratosis, psoriasis, and related forms of hyperproliferative and pre-cancerous skin diseases, by administration of an inhibitor of β-catenin. This disclosure also provides alpha helix mimetic β-catenin inhibitor compounds, and compositions comprising an inhibitor of β-catenin.

### BRIEF DESCRIPTION OF THE FIGURES

FIGS. 1A-1B. qPCR results showing the effect of test article Compound C on Elafin gene expression levels in the psoriatic tissue model following (A) two topical applications (2× at t=0, 24) and (B) four topical applications (4× at t=0, 24, 48, and 72 hr) ±SEM, N=3. Compound C is (6S,9S,9aS)—N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide.

FIGS. 2A-2B. qPCR results showing the effect of test article Compound C on HBD-2 gene expression levels in the psoriatic tissue model following (A) two topical applications (2× at t=0, 24) and (B) four topical applications (4× at t=0, 24, 48, and 72 hr) ±SEM, N=3.

FIGS. 3A-3B. qPCR results showing the effect of test article Compound C on psoriasin gene expression levels in the psoriatic tissue model following (A) two topical applications (2× at t=0, 24) and (B) four topical applications (4× at t=0, 24, 48, and 72 hr) ±SEM, N=3.

FIGS. 4A-4B. qPCR results showing the effect of test article Compound C on Ki67 gene expression levels in the psoriatic tissue model following (A) two topical applications (2× at t=0, 24) and (B) four topical applications (4× at t=0, 24, 48, and 72 hr) ±SEM, N=3.

FIGS. 5A-5B. qPCR results showing the effect of test article Compound C on p63 gene expression levels in the psoriatic tissue model following (A) two topical applications (2× at t=0, 24) and (B) four topical applications (4× at t=0, 24, 48, and 72 hr) ESEM, N=3.

### DETAILED DESCRIPTION OF THE DISCLOSURE

Recently, non-peptide compounds have been developed which mimic the secondary structure of reverse-turns found in biologically active proteins or peptides. For example, U.S. Pat. No. 5,440,013 and published PCT Applications Nos. WO94/03494, WO01/00210A1, and WO01/16135A2 each disclose conformationally constrained, non-peptidic compounds, which mimic the three-dimensional structure of reverse-turns. In addition, U.S. Pat. No. 5,929,237 and its continuation-in-part U.S. Pat. No. 6,013,458, disclose conformationally constrained compounds which mimic the secondary structure of reverse-turn regions of biologically active peptides and proteins. In relation to reverse-turn mimetics, conformationally constrained compounds have been disclosed which mimic the secondary structure of alpha-helix regions of biologically active peptide and proteins in WO2007/056513 and WO2007/056593.

This disclosure provides novel compounds, pharmaceutical compositions and methods of treatment for hyperproliferative and pre-cancerous skin conditions including actinic keratosis and psoriasis. The inventors have determined that inhibiting β-catenin signaling is an effective approach to the treatment of such conditions.

The structures and compounds of the alpha helix mimetic β-catenin inhibitors of this invention are disclosed in WO 2010/044485, WO 2010/128685, WO 2009/148192, and US 2011/0092459, each of which is incorporated herein by reference in its entirety. These compounds have now been found to be useful in the treatment of actinic keratosis and psoriasis, and related forms of hyperproliferative and pre-cancerous skin disease. While not wishing to be bound, the effectiveness of these compounds in treating these conditions is based in part on the ability of these compounds to block TCF4/β-catenin transcriptional pathway by inhibiting cyclic AMP response-element binding protein (CBP), thus altering wnt pathway signaling, which has been found to improve outcomes.

The preferable structure of the alpha helix mimetic β-catenin inhibitors of this invention have the following formula (I):

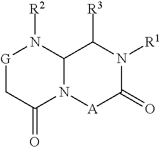

wherein

### A is —CHR⁷—,

wherein

R⁷ is optionally substituted arylalkyl, optionally substituted heteroarylalkyl, optionally substituted cycloalkylalkyl or optionally substituted heterocycloalkylalkyl; G is —NH—, —NR⁶—, or —O—

wherein

R⁶ is lower alkyl or lower alkenyl;

### R¹ is -Ra-R¹⁰;

wherein

Ra is optionally substituted lower alkylene and

R¹⁰ is optionally substituted bicyclic fused aryl or optionally substituted bicyclic fused heteroaryl;

### R² is —(CO)—NH-Rb-R²⁰,

wherein

Rb is bond or optionally substituted lower alkylene; and

R²⁰ is optionally substituted aryl or optionally substituted heteroaryl; and

R³ is C₁₋₄ alkyl.
These compounds are especially useful in the prevention and/or treatment of hyperproliferative and pre-cancerous skin conditions including actinic keratosis and psoriasis.

The more preferable structure of the alpha helix mimetic β-catenin inhibitors of this invention have the following substituents in the above-mentioned formula (I):

### A is —CHR⁷—,

wherein

R⁷ is arylalkyl optionally substituted with hydroxyl or C₁₋₄ alkyl;

### G is —NH—, —NR⁶—, or —O—

wherein

R⁶ is C₁₋₄ alkyl or C₁₋₄ alkenyl;

### R¹ is -Ra-R¹⁰;

wherein

Ra is C₁₋₄ alkylene and

R¹⁰ is bicyclic fused aryl or bicyclic fused heteroaryl, optionally substituted with halogen or amino;

### R² is —(CO)—NH-Rb-R²⁰,

wherein

Rb is bond or C₁₋₄ alkylene; and

R²⁰ is aryl or heteroaryl; and

R³ is C₁₋₄ alkyl.
These compounds are especially useful in the prevention and/or treatment of hyperproliferative and pre-cancerous skin conditions including actinic keratosis and psoriasis.

The most preferable alpha helix mimetic β-catenin inhibitors of this invention are as follows:
(6S,9S)—N-benzyl-6-(4-hydroxybenzyl)-2,9-dimethyl-8-(naphthalen-1-ylmethyl)-4,7-dioxooctahydro-1H-pyrazino[2,1-c][1,2,4]triazine-1-carboxamide,
(6S,9S)-2-allyl-N-benzyl-6-(4-hydroxybenzyl)-9-methyl-8-(naphthalen-1-ylmethyl)-4,7-dioxooctahydro-1H-pyrazino[2,1-c][1,2,4]triazine-1-carboxamide,
(6S,9S)—N-benzyl-6-(4-hydroxybenzyl)-9-methyl-8-(naphthalen-1-ylmethyl)-4,7-dioxohexahydropyrazino[2,1-c][1,2,4]oxadiazine-1(6H)-carboxamide,
(6S,9S)-8-((2-aminobenzo[d]thiazol-4-yl)methyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide,
(6S,9S)-2-allyl-N-benzyl-6-(4-hydroxybenzyl)-9-methyl-4,7-dioxo-8-(quinolin-8-ylmethyl)octahydro-1H-pyrazino[2,1-c][1,2,4]triazine-1-carboxamide,
4-(((6S,9S)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate,
4-(((6S,9S)-1-(benzylcarbamoyl)-2,9-dimethyl-8-(naphthalen-1-ylmethyl)-4,7-dioxooctahydro-1H-pyrazino[2,1-c][1,2,4]triazin-6-yl)methyl)phenyl dihydrogen phosphate,
sodium 4-(((6S,9S)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl phosphate,
sodium 4-(((6S,9S)-1-(benzylcarbamoyl)-2,9-dimethyl-4,7-dioxo-8-(naphthalen-8-ylmethyl)octahydro-1H-pyrazino[2,1-c][1,2,4]triazin-6-yl)methyl)phenyl phosphate,
(6S,9S)-2-allyl-6-(4-hydroxybenzyl)-9-methyl-4,7-dioxo-N—((R)-1-phenylethyl)-8-(quinolin-8-ylmethyl)octahydro-1H-pyrazino[2,1-c][1,2,4]triazine-1-carboxamide,
(6S,9S)-2-allyl-6-(4-hydroxybenzyl)-9-methyl-4,7-dioxo-N—((S)-1-phenylethyl)-8-(quinolin-8-ylmethyl)octahydro-1H-pyrazino[2,1-c][1,2,4]triazine-1-carboxamide,
(6S,9S)—N-benzyl-6-(4-hydroxy-2,6-dimethylbenzyl)-2,9-dimethyl-4,7-dioxo-8-(quinolin-8-ylmethyl)octahydro-1H-pyrazino[2,1-c][1,2,4]triazine-1-carboxamide,
(6S,9S)-8-(benzo[b]thiophen-3-ylmethyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)-8-(benzo[c][1,2,5]thiadiazol-4-ylmethyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-8-(isoquinolin-5-ylmethyl)-2,9-dimethyl-4,7-dioxooctahydro-1H-pyrazino[2,1-c][1,2,4]triazine-1-carboxamide,
(6S,9S)—N-benzyl-8-((5-chlorothieno[3,2-b]pyridin-3-yl)methyl)-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-2,9-dimethyl-4,7-dioxo-8-(quinoxalin-5-ylmethyl)octahydro-1H-pyrazino[2,1-c][1,2,4]triazine-1-carboxamide, and
(6S,9S)-6-(4-hydroxybenzyl)-2,9-dimethyl-4,7-dioxo-8-(quinolin-8-ylmethyl)-N-(thiophen-2-ylmethyl)octahydro-1H-pyrazino[2,1-c][1,2,4]triazine-1-carboxamide.
   These compounds are especially useful in the prevention and/or treatment of hyperproliferative and pre-cancerous skin conditions including actinic keratosis and psoriasis.

In a most preferred embodiment, the compound is:
4-(((6S,9S,9aS)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate (Compound A), or
(6S,9S,9aS)—N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide (Compound C).
   These compounds are especially useful in the prevention and/or treatment of hyperproliferative and pre-cancerous skin conditions including actinic keratosis and psoriasis.

A “β-catenin inhibitor” is a substance that can reduce or prevent β-catenin activity. β-catenin activities include translocation to the nucleus, binding with TCF (T cell factor) transcription factors, and coactivating TCF transcription factor-induced transcription of TCF target genes. A “β-catenin inhibitor” can also interfere with the interaction of CBP and β-catenin. Thus, a β-catenin inhibitor inhibits or reduces CBP/β-catenin signaling and activity of the CBP/β-catenin signaling pathway, including reduction of one or more downstream signaling events.

Disclosed herein are alpha helix mimetic β-catenin inhibitor compounds for treatment of hyperproliferative and pre-cancerous skin conditions including actinic keratosis and psoriasis.

### Diseases

A “hyperproliferative” disease or condition is characterized by excessive or unwanted cellular proliferation.

A “pre-cancerous” disease or condition is characterized by cellular dysplasia and altered cellular growth associated with progression to a neoplastic or cancerous state.

“Treatment” refers to clinical intervention in an attempt to alter the disease course of the individual or cell being treated, and can be performed during the course of clinical pathology. Therapeutic effects of treatment include without limitation, preventing recurrence of disease, alleviation of symptoms, diminishment of any direct or indirect pathological consequences of the disease, decreasing the rate of disease progression, amelioration or palliation of the disease state, and remission or improved prognosis.

As used herein, the terms “therapeutically effective amount” and “effective amount” are used interchangeably to refer to an amount of a composition of the invention that is sufficient to result in the prevention of the development or onset of hyperproliferative and pre-cancerous skin conditions including actinic keratosis and psoriasis, or one or more symptoms thereof, to enhance or improve the effect(s) of another therapy, and/or to ameliorate one or more symptoms of hyperproliferative and pre-cancerous skin conditions including actinic keratosis and psoriasis. For a subject suffering from actinic keratosis, a preferred therapeutically effective amount is an amount effective to reduce symptoms of keratosis, such as a reduction in the presence or formation of skin lesions. For a subject suffering from psoriasis, a preferred therapeutically effective amount is an amount effective to reduce symptoms of psoriasis, such as a reduction in the presence or formation of skin plaques.

A therapeutically effective amount can be administered to a patient in one or more doses sufficient to palliate, ameliorate, stabilize, reverse or slow the progression of the disease, or otherwise reduce the pathological consequences of the disease, or reduce the symptoms of the disease. The amelioration or reduction need not be permanent, but may be for a period of time ranging from at least one hour, at least one day, or at least one week or more. The effective amount is generally determined by the physician on a case-by-case basis and is within the skill of one in the art. Several factors are typically taken into account when determining an appropriate dosage to achieve an effective amount. These factors include age, sex and weight of the patient, the condition being treated, the severity of the condition, as well as the route of administration, dosage form and regimen and the desired result.

As used herein, the terms “subject” and “patient” are used interchangeably and refer to an animal, preferably a mammal such as a non-primate (e.g., cows, pigs, horses, cats, dogs, rats etc.) and a primate (e.g., monkey and human), and most preferably a human.

The alpha helix mimetic β-catenin inhibitors described herein are useful to prevent or treat disease. Specifically, the disclosure provides for both prophylactic and therapeutic methods of treating a subject at risk of (or susceptible to) hyperproliferative and pre-cancerous skin conditions including actinic keratosis and psoriasis. Accordingly, the present methods provide for the prevention and/or treatment of these conditions in a subject by administering an effective amount of alpha helix mimetic β-catenin inhibitors to a subject in need thereof. For example, a subject can be administered the alpha helix mimetic β-catenin inhibitors in an effort to improve one or more of the factors of a hyperproliferative and pre-cancerous skin condition including actinic keratosis and psoriasis.

As used herein, “actinic keratosis” refers to a skin condition characterized by development of thickened skin lesions. Progressive development of these lesions occurs when skin is exposed to the sun constantly and thick, scaly, or crusty areas appear. The scaly or crusty portion is dry and rough. The lesions start out as flat scaly areas and later grow into a tough, wart-like area. Untreated lesions have up to twenty percent risk of progression to squamous cell carcinoma. People who take immunosuppressive drugs, such as organ transplant patients, are more likely to develop actinic keratoses that lead to skin cancer.

As used herein “psoriasis” refers to a hyperproliferative disease that affects the skin, resulting in excessive proliferation of skin cells. Depending on the severity and location of outbreaks, individuals may experience significant physical discomfort and some disability. There are five types of psoriasis: plaque, guttate, inverse, pustular, and erythrodermic.

In plaque psoriasis, skin rapidly accumulates at localized sites, which gives the skin a silvery-white appearance. Plaques frequently occur on the skin of the elbows and knees, but can affect any area, including the scalp, palms of hands and soles of feet, and genitals. This is the most common form of psoriasis.

Guttate psoriasis is characterized by numerous small, scaly, red or pink, teardrop-shaped lesions. These numerous spots of psoriasis appear over large areas of the body, primarily the trunk, but also the limbs and scalp.

Inverse psoriasis appears as smooth inflamed patches of skin. It occurs in skin folds, particularly between the thigh and groin, the armpits, under an overweight abdomen, and under the breasts. It is aggravated by friction and sweat, and is makes the skin vulnerable to fungal infections.

Pustular psoriasis appears as raised bumps that are filled with noninfectious pus (pustules). The skin under and surrounding the pustules is red and tender. Pustular psoriasis can be localized, commonly to the hands and feet (palmoplantar pustulosis), or generalized with widespread patches occurring randomly on any part of the body.

Erythrodermic psoriasis involves widespread inflammation and exfoliation of the skin over most of the body surface. It may be accompanied by severe itching, swelling and pain. It is often the result of an exacerbation of unstable plaque psoriasis, particularly following the abrupt withdrawal of systemic treatment. This form of psoriasis can be fatal, as the extreme inflammation and exfoliation disrupt the body's ability to regulate temperature and for the skin to perform barrier functions.

This invention provides treatments for hyperproliferative and pre-cancerous skin conditions, including actinic keratosis and psoriasis, by administration of a β-catenin inhibitor. The invention also encompasses methods where the β-catenin inhibitor compound is given in combination therapy. That is, the compound can be used in conjunction with, but separately from, other agents useful in treating hyperproliferative and pre-cancerous skin conditions, including actinic keratosis and psoriasis. In these combination methods, the compound will generally be given in a daily dose of 1-100 mg/kg body weight daily in conjunction with other agents. The other agents generally will be given in the amounts used therapeutically. The specific dosing regime, however, will be determined by a physician using sound medical judgment.

Treatment of actinic keratosis refers to the administration of a compound or combination described herein to treat a subject suffering from actinic keratosis. One outcome of the treatment of actinic keratosis is to reduce formation of excessive connective tissue. Another outcome of the treatment of actinic keratosis is to reduce or prevent the development of skin lesions. Still another outcome of the treatment of actinic keratosis is to prevent or inhibit the development of skin cancer.

Treatment of psoriasis refers to the administration of a compound or combination described herein to treat a subject suffering from psoriasis. One outcome of the treatment of psoriasis is to reduce formation of skin plaques. Another outcome of the treatment of psoriasis is to reduce or prevent skin itching and/or flaking. Still another outcome of the treatment of psoriasis is to reduce psoriasis-related inflammation.

The alpha helix mimetic β-catenin inhibitors described herein can be incorporated into pharmaceutical compositions for administration, singly or in combination, to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Any suitable route of administration may be employed for providing a mammal, especially a human, with an effective dose of a compound described herein. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The effective dosage of active ingredient employed may vary depending on the particular compound employed, the mode of administration, the condition being treated and the severity of the condition being treated. Such dosage may be ascertained readily by a person skilled in the art.

When treating or controlling actinic keratosis, psoriasis and/or other conditions for which compounds described herein are indicated, generally satisfactory results are obtained when the compounds described herein are administered at a daily dosage of from about 0.01 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams. In the case of a 70 kg adult human, the total daily dose will generally be from about 1 milligram to about 500 milligrams. For a particularly potent compound, the dosage for an adult human may be as low as 0.1 mg. In some cases, the daily dose may be as high as 1 gram. The dosage regimen may be adjusted within this range or even outside of this range to provide the optimal therapeutic response.

Oral administration will usually be carried out using tablets or capsules. Examples of doses in tablets and capsules are 0.1 mg, 0.25 mg, 0.5 mg, 1 mg, 2 mg, 5 mg, 10 mg, 15 mg, 20 mg, 25 mg, 30 mg, 40 mg, 50 mg, 100 mg, 200 mg, 250 mg, 300 mg, 400 mg, 500 mg, and 750 mg. Other oral forms may also have the same or similar dosages.

Also described herein are pharmaceutical compositions which comprise a compound described herein and a pharmaceutically acceptable carrier. The pharmaceutical compositions described herein comprise a compound described herein or a pharmaceutically acceptable salt as an active ingredient, as well as a pharmaceutically acceptable carrier and optionally other therapeutic ingredients. A pharmaceutical composition may also comprise a prodrug, or a pharmaceutically acceptable salt thereof, if a prodrug is administered.

The compositions can be suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds described herein can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions as oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharm. Res. 3(6):318 (1986).

Pharmaceutical formulations adapted for topical administration may be formulated as ointments, creams, suspensions, lotions, powders, solutions, pastes, gels, sprays, aerosols, or oils. For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in oil base.

Compounds described herein may also be administered parenterally. Solutions or suspensions of these active compounds can be prepared in water suitably mixed with a surfactant or mixture of surfactants such as hydroxypropylcellulose, polysorbate 80, and mono and diglycerides of medium and long chain fatty acids. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

The present disclosure is further illustrated by the following non-limiting examples.

### Examples

The purpose of this study was to investigate the effects of a test compound, Compound C, on the structure and gene expression of a human cell based psoriatic tissue model. Compound C is (6S,9S,9aS)—N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide, an alpha helix mimetic β-catenin inhibitor compound.

The psoriasis tissue model (SOR-300-FT; MatTek Corp., Ashland, Mass.) is a highly differentiated in vitro psoriatic tissue comprised of normal, human-derived keratinocytes and psoriatic fibroblasts which have been cultured to form a highly differentiated model of psoriatic tissue. Morphologically, the psoriatic tissue is of uniform thickness and is very similar to native psoriatic tissue in that it expresses increased levels of Ki67+ cells (hyperproliferation) and psoriasis-associated proteins such as psoriasin, elafin and human beta-defensin-2 (HBD-2) (Am. J. Pathol. 173:815-23, 2008). SOR-300-FT tissues (surface area=0.6 cm²) were cultured on microporous membrane (pore size=0.4 μm) cell culture inserts. All psoriatic tissues were grown at the air liquid interface (ALI) in which the apical tissue layer is exposed to air. Use of the ALI induces the tissues to attain skin-like differentiation and allows direct topical application of test materials (similar to in vivo exposure).

Calcipotriol at 2.5, 0.25 and 0.025 μg/ml in medium was used as positive control. Ultrapure water was used as the vehicle control. The test article Compound C was applied to the basolateral (by additions to the culture medium) and apical (50 μl) side of the tissues. The effect of each three concentrations of the test article on tissue structure was examined using H&E stained histological slides. For gene analysis, RNA was isolated using standard RNA isolation protocol (MatTek Corporation). Isolated RNA was quantified and the integrity of the isolated RNA was checked. Quantitative RT-PCR was performed to determine expression levels of 5 psoriatic associated genes (elafin, HBD-2, psoriasin, p63, and Ki67).

SOR-300-FT™ tissues were transferred to 6-well plates containing 0.9 ml of pre-warmed assay medium and equilibrated to standard culture conditions (37° C., 5% CO₂) for 1 hour. After the 1 hr equilibration, the tissues were re-fed with fresh medium as follow: 1) for the 24 hr time point, tissues were feed with 0.9 ml of medium and 2) for time points >24 hr, tissues were fed with 5 ml of culture medium by placing the cell culture inserts on top of the washers (Part # EPI-WSHR, MatTek Corporation). Next, 50 μl of the test article was applied topically to the psoriatic tissues (n=3) and the test article was added to the culture medium at concentrations of 100 μM, 30 μM, and 5 μM. At times 48 and 72 hours: a) the tissues were rinsed topically 3× with 300-400 μL of PBS, b) the inserts containing the tissues were held tightly with sterile forceps and the test article was rinsed gently by immersing the insert into PBS and decant medium from insert and c) fresh test article was re-applied to the tissue immediately after rinsing and decanting (50 μl, topically). Analysis was performed at t=48 hr (2× repeat applications) and t=96 hr (4× applications).

After t=48 (2× repeat exposure) and t=96 hr (4× exposure), N=3 tissues/treatment were used for RNA isolation for biomarker identification (gene expression levels). After the t=48 and t=96 hr, N=1 tissue/treatment was fixed in 10% formalin for histological analysis.

RNA Isolation: RNA was isolated from the tissues following MatTek's standardized RNA isolation protocol. The concentration, integrity, and purity of RNA was assessed using Experion System (Bio-Rad).

qPCR: cDNA was generated using the RT2 First Strand Kit (Qiagen, cat#330401). Relative gene expression was measured using RT2 SYBR Green qPCR Mastermix (Qiagen, cat#330502) and Qiagen RT2 primers. Analysis was carried out using Bio-Rad CFX software.

Effect of Test Drugs on Tissue Structure.

Microscopic observations of histology samples were performed and effect of treatment on tissue morphology such as apical surface disruption, structural changes, and abnormal tissue staining was assessed.

Test Article Compound C, 100 μM: Microscopic observation of the tissue histology following 2× or 4× repeat exposures to the test article showed structural damage to the differentiated cell layer (suprabasal to apical layers).

Test Article Compound C, 30 μM: Microscopic observation of the tissue histology following 2× repeat exposures to the test article showed minor structural damage at 48 hr. At 96 hr following 4× exposures to the test article, the suprabasal and apical layer was sloughed off leaving behind the basal cells still attached to the dermal component of the tissue. This sloughing suggests removal of psoriatic plaques.

Test Article Compound C, 5 μM: Microscopic observation of the tissue histology following 2× repeat exposures showed minimal effect on tissue structure. However, following 4× repeat exposures to the test article, the parabasal-apical layer of the tissue was sloughed off. This sloughing suggests removal of psoriatic plaques. In addition, the basal cells were intact and tissue regeneration was occurring.

Calcipotriol—Positive Control: Microscopic observation of the tissue histology following 2× or 4× repeat exposures to different concentrations of the positive control showed minimal-to-no evidence of structural damage or significant changes to tissue morphology.

Culture medium—Negative Control: Normal tissue histology was observed over the entire experiment.

Effect of Test Drugs on Biomarker Genes.

qPCR was performed to quantify the relative gene expression levels of 5 psoriatic associated gene biomarkers: 1) human beta defensin 2 (HBD-2), 2) psoriasin, 3) elafin and 4) p63, and 5) Ki67.

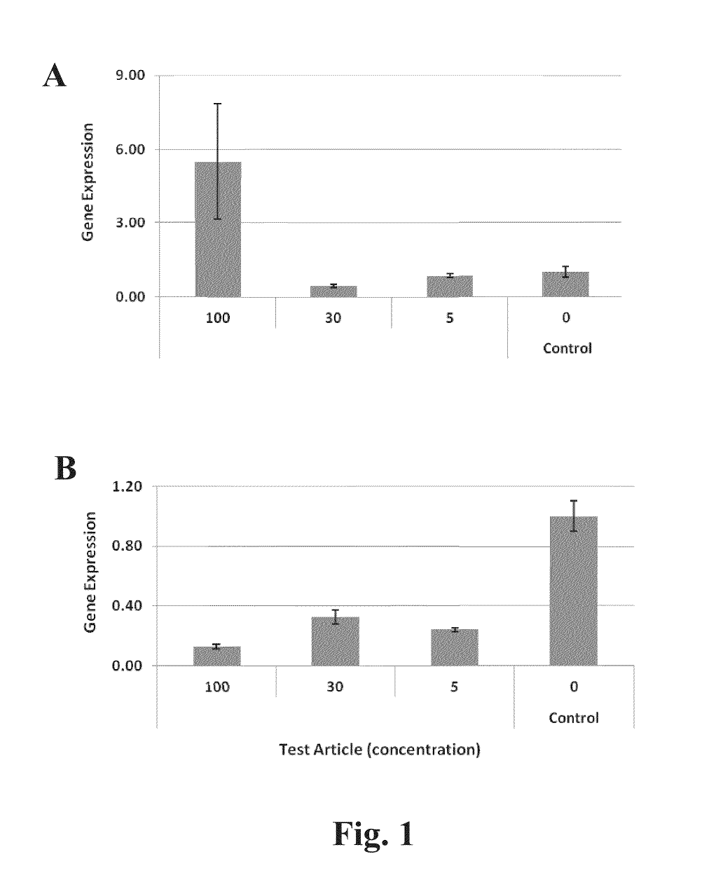

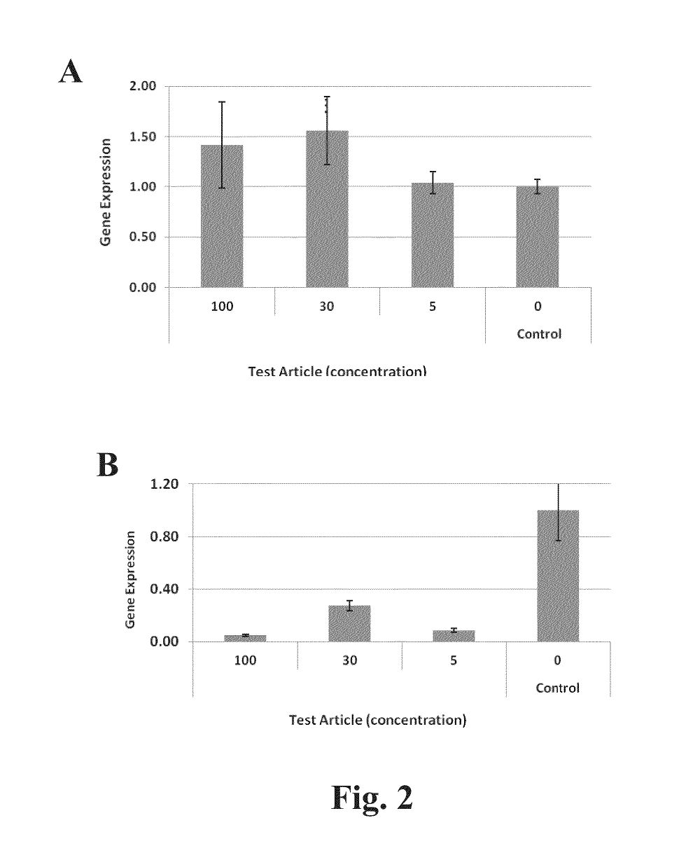

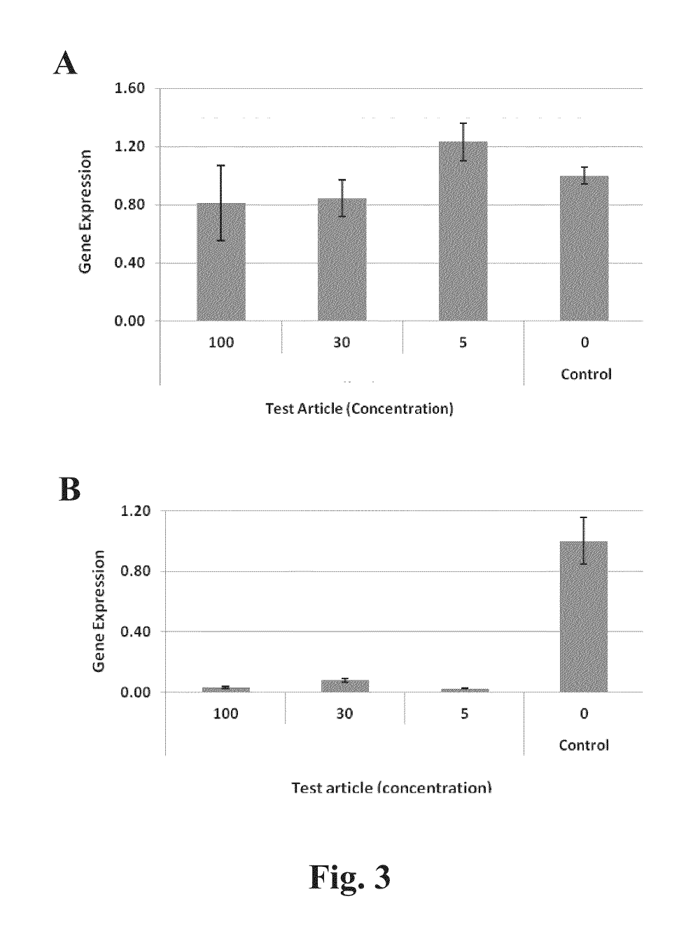

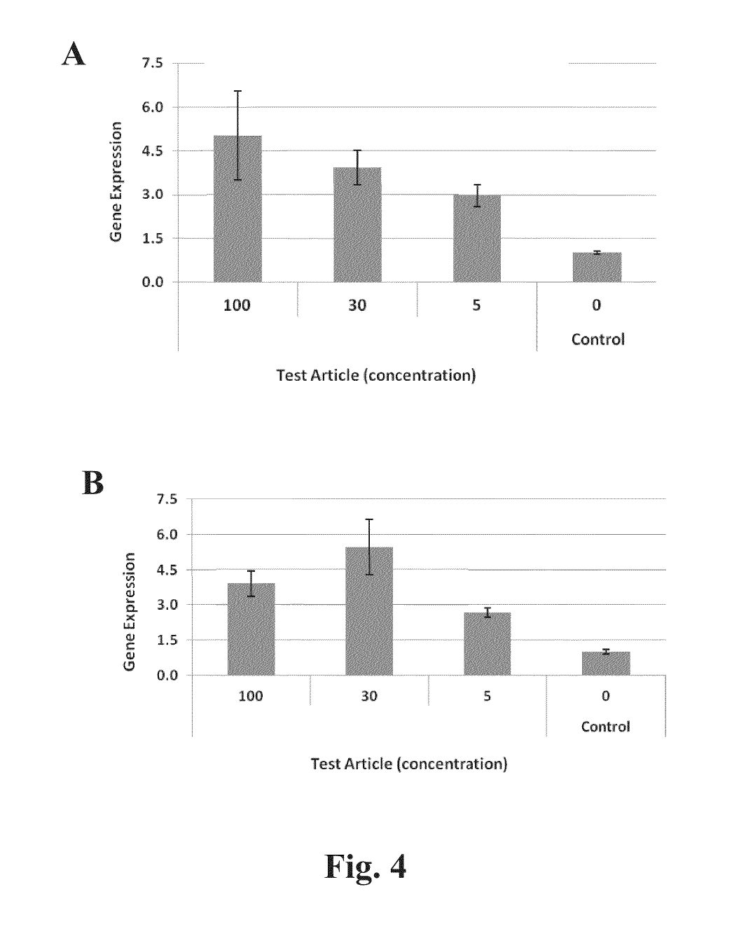

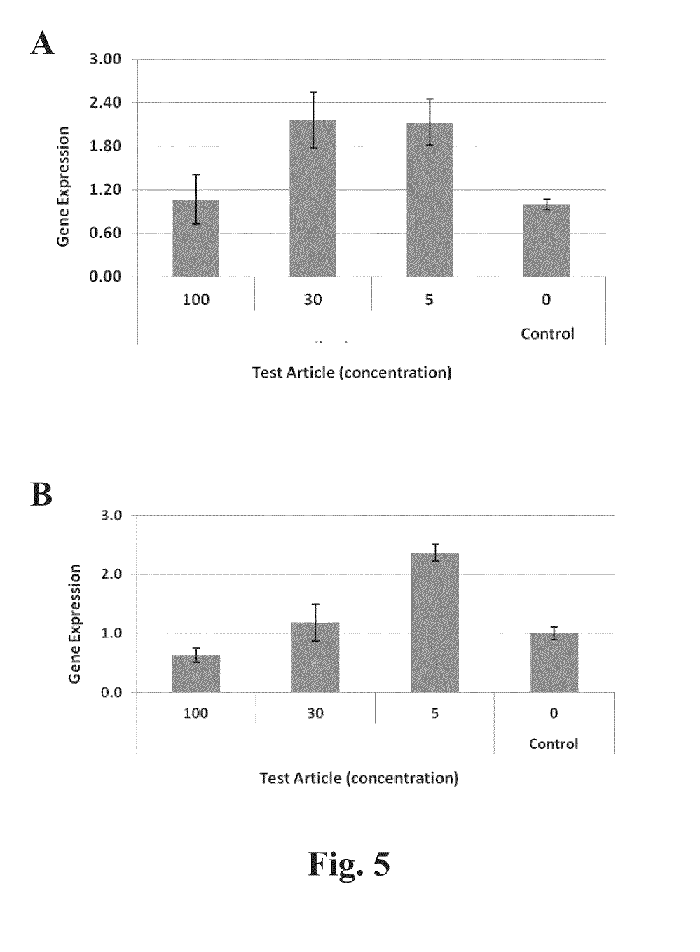

Test Article Compound C, 100 μM: The 100 μM concentration downregulated (≧2 fold) the expression of elafin, HBD-2, and psoriasin following repeat (4×) applications over a 96 hr exposure period (FIGS. 1B, 2B, 3B). Note: 100 μM concentration of test article did show a slight downregulation (1.6 fold) of the p63 gene following 2× repeat applications (FIG. 5A). No down regulation of Ki67 gene expression was noted for the test article at this concentration (FIGS. 4A-B; Tables 1 and 2).

Test Article Compound C, 30 μM: The 30 μM concentration downregulated (≧2 fold) the expression of elafin, HBD-2, and psoriasin following repeat (4×) applications over a 96 hr exposure period (FIGS. 1B, 2B, 3B). In addition, this concentration also showed a 2.2 fold reduction in elafin gene expression level at time 48 hr (FIG. 1A). Note: the 30 μM concentration of Compound C test article did not show downregulation of p63 and Ki67 genes following multiple repeat applications (2× or 4×; FIGS. 4A-B and 5A-B; Tables 1 and 2).

Test Article Compound C, 5 μM: The 5 μM concentration downregulated (≧2 fold) the expression of elafin, HBD-2, and psoriasin following 4× repeat applications over a 96 hr exposure time (FIGS. 1B, 2B, 3B). Note: the 5 μM concentration of Compound C test article did not show downregulation of the p63 and Ki67 genes following multiple repeat applications (2× or 4×; FIGS. 4A-B and 5A-B; Tables 1 and 2).

Calcipotriol (Positive Control): The 2.5 μg/ml concentration of the drug downregulated (≧2 fold) the expression of all tested psoraisis-associated genes (elafin, HBD-2, and psoriasin, p63, and Ki 67) following 2× repeat applications (Table 1). After 4× repeat applications, a significant reduction for the elafin, HBD-2, and psoriasin genes was noted (Table 2); the p63 and Ki67 genes showed a 1.7 and a 1.6 fold decrease, respectively (Table 2). Furthermore, the 0.25 ug/ml concentration of calciptoriol also showed downregulation (≧2 fold) of HBD2 and p63 gene expression levels following 2× application over a 48 hr exposure period. The lowest concentration of calcipotriol (0.025 ug/ml) showed downregulation (≧2 fold) in the expression levels of elafin, HBD-2, and psoriasin following 4× repeat applications but not after 2× repeat applications.

TABLE 1
Fold change in gene expression levels following
repeat (2X at 48 hr) applications of Compound C and
calcipotriol on the psoriasis tissue model. Downregulated gene
expression levels (>2 fold) are shaded.

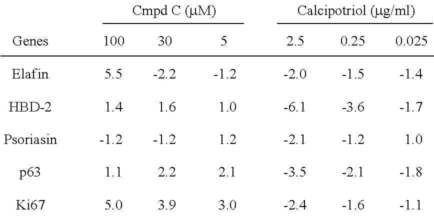

TABLE 2
Fold change in gene expression levels following
repeat (4X at 96 hr) applications of Compound C and
calcipotriol in the psoriasis tissue model. Downregulated gene
expression levels (>2 fold) are shaded.

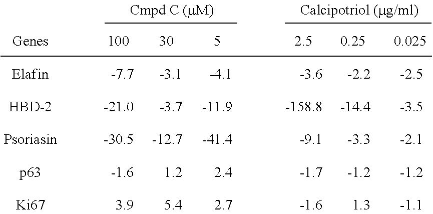

Treatment of tissues with Compound C showed downregulation of psoriasis-related gene markers, elafin, HBD-2, and psoriasin. In addition, treatment with Compound C led to sloughing of apical layer, which is associated with removal of psoriatic plaques in vivo, and tissue regeneration. Hence, Compound C, an exemplary alpha helix mimetic β-catenin inhibitor compound of the invention, is effective for the treatment of psoriasis.

1. An alpha helix mimetic β-catenin inhibitor compound for the treatment of a hyperproliferative or pre-cancerous skin condition, having the following formula (I): 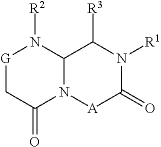
wherein:
A is —CHR⁷—,
wherein R⁷ is hydrogen, optionally substituted alkyl, optionally substituted alkenyl, optionally substituted alkynyl, optionally substituted arylalkyl, optionally substituted heteroarylalkyl, optionally substituted cycloalkylalkyl, optionally substituted heterocycloalkylalkyl, optionally substituted aryl, optionally substituted heteroaryl, optionally substituted cycloalkyl or optionally substituted heterocycloalkyl;
G is —NH—, —NR⁶—, —O—, —CHR⁶— or —C(R⁶)₂—,
wherein R⁶ is independently selected from optionally substituted alkyl, optionally substituted alkenyl and optionally substituted alkynyl;
R¹ is optionally substituted arylalkyl, optionally substituted heteroarylalkyl, optionally substituted cycloalkylalkyl or optionally substituted heterocycloalkylalkyl;
R² is —W²¹—W²²-Rb-R²⁰,
wherein W²¹ is —(CO)— or —(SO₂)—; W²² is bond, —O—, —NH— or optionally substituted lower alkylene; Rb is bond or optionally substituted lower alkylene; and R²⁰ is optionally substituted alkyl, optionally substituted alkenyl, optionally substituted alkynyl, optionally substituted aryl, optionally substituted heteroaryl, optionally substituted cycloalkyl or optionally substituted heterocycloalkyl; and
R³ is optionally substituted alkyl, optionally substituted alkenyl or optionally substituted alkynyl;
or a pharmaceutically acceptable salt thereof.

2. The compound of claim 1, selected from:
(6S,9S)—N-benzyl-6-(4-hydroxybenzyl)-2,9-dimethyl-8-(naphthalen-1-ylmethyl)-4,7-dioxooctahydro-1H-pyrazino[2,1-c][1,2,4]triazine-1-carboxamide,
(6S,9S)-2-allyl-N-benzyl-6-(4-hydroxybenzyl)-9-methyl-8-(naphthalen-1-ylmethyl)-4,7-dioxooctahydro-1H-pyrazino[2,1-c][1,2,4]triazine-1-carboxamide,
(6S,9S)—N-benzyl-6-(4-hydroxybenzyl)-9-methyl-8-(naphthalen-1-ylmethyl)-4,7-dioxohexahydropyrazino[2,1-c][1,2,4]oxadiazine-1(6H)-carboxamide,
(6S,9S)-8-((2-aminobenzo[d]thiazol-4-yl)methyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide,
(6S,9S)-2-allyl-N-benzyl-6-(4-hydroxybenzyl)-9-methyl-4,7-dioxo-8-(quinolin-8-ylmethyl)octahydro-1H-pyrazino[2,1-c][1,2,4]triazine-1-carboxamide,
4-(((6S,9S)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate,
4-(((6S,9S)-1-(benzylcarbamoyl)-2,9-dimethyl-8-(naphthalen-1-ylmethyl)-4,7-dioxooctahydro-1H-pyrazino[2,1-c][1,2,4]triazin-6-yl)methyl)phenyl dihydrogen phosphate,
sodium 4-(((6S,9S)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl phosphate,
sodium 4-(((6S,9S)-1-(benzylcarbamoyl)-2,9-dimethyl-4,7-dioxo-8-(naphthalen-8-ylmethyl)octahydro-1H-pyrazino[2,1-c][1,2,4]triazin-6-yl)methyl)phenyl phosphate,
(6S,9S)-2-allyl-6-(4-hydroxybenzyl)-9-methyl-4,7-dioxo-N—((R)-1-phenylethyl)-8-(quinolin-8-ylmethyl)octahydro-1H-pyrazino[2,1-c][1,2,4]triazine-1-carboxamide,
(6S,9S)-2-allyl-6-(4-hydroxybenzyl)-9-methyl-4,7-dioxo-N—((S)-1-phenylethyl)-8-(quinolin-8-ylmethyl)octahydro-1H-pyrazino[2,1-c][1,2,4]triazine-1-carboxamide,
(6S,9S)—N-benzyl-6-(4-hydroxy-2,6-dimethylbenzyl)-2,9-dimethyl-4,7-dioxo-8-(quinolin-8-ylmethyl)octahydro-1H-pyrazino[2,1-c][1,2,4]triazine-1-carboxamide,
(6S,9S)-8-(benzo[b]thiophen-3-ylmethyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)-8-(benzo[c][1,2,5]thiadiazol-4-ylmethyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-8-(isoquinolin-5-ylmethyl)-2,9-dimethyl-4,7-dioxooctahydro-1H-pyrazino[2,1-c][1,2,4]triazine-1-carboxamide,
(6S,9S)—N-benzyl-8-((5-chlorothieno[3,2-b]pyridin-3-yl)methyl)-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-2,9-dimethyl-4,7-dioxo-8-(quinoxalin-5-ylmethyl)octahydro-1H-pyrazino[2,1-c][1,2,4]triazine-1-carboxamide, and
(6S,9S)-6-(4-hydroxybenzyl)-2,9-dimethyl-4,7-dioxo-8-(quinolin-8-ylmethyl)-N-(thiophen-2-ylmethyl)octahydro-1H-pyrazino[2,1-c][1,2,4]triazine-1-carboxamide.

3. The compound of claim 1, selected from:
4-(((6S,9S,9aS)-1-(benzylcarbamoyl)-2,9-dimethyl-4,7-dioxo-8-(quinolin-8-ylmethyl) octahydro-1H-pyrazino[2,1-c][1,2,4]triazin-6-yl)methyl)phenyl dihydrogen phosphate, and
(6S,9S,9aS)—N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide.

4. A pharmaceutical composition comprising the compound of claim 1.

5. A method of treatment for a hyperproliferative or pre-cancerous skin condition, comprising administering an effective amount of the compound of claim 1 to a patient in need thereof.

6. The method of claim 5, wherein the skin condition is actinic keratosis.

7. The method of claim 5, wherein the skin condition is psoriasis.

8. A method of inhibiting the development of skin cancer subsequent to actinic keratosis, comprising administering an effective amount of the compound of claim 1 to a patient in need thereof.
